(19) United States
(12) Patent Application Publication
(10) Pub. No.: US 2013/0165366 A1
(43) Pub. Date: Jun. 27, 2013

(54) Fragrance Tablet and Method

(71) Applicant: Jeff Slaboden, Watsonville, CA (US)

(72) Inventor: Jeff Slaboden, Watsonville, CA (US)

(21) Appl. No.: 13/335,933

(22) Filed: Dec. 23, 2011

(51) Int. Cl. C11D 17/00; C11D 3/60; A61L 9/012

(52) U.S. Cl. 510/276; 512/4; 510/513; 510/516

(57) ABSTRACT

A fragrance tablet having a fragrance selected from the group consisting of synthetic fragrances, natural fragrances and essential oils; and a fragrance tablet delivery device, wherein said fragrance tablet delivery device is water soluble and related method is disclosed. The fragrance tablet dissolves in wash water and mixes with non-fragranced laundry detergent to create a desired scent.

### REFERENCE TO PENDING APPLICATIONS

This application does not claim the benefit of and pending U.S. patent application.

### REFERENCE TO MICROFICHE APPENDIX

This application is not referenced in any microfiche appendix.

### BACKGROUND OF THE INVENTION

The present invention is generally directed toward fragrance tablet, and more specifically toward fragrance tablet used with non-fragranced laundry detergent to produce a desired scent, and related method.

Currently in the United States over 35 billon loads of laundry are washed annually. These loads of laundry are washed using a variety of laundry detergent, including fragranced and non-fragranced detergent. The fragranced detergent, while enjoyable to some users, can be overbearing and not pleasing to other users. Further, commonly used fragrances can cause allergic reactions or skin irritation to users.

Additionally, the prior art fragranced laundry detergent is sold in containers suitable to hold multiple ounces of the laundry detergent. This causes a disadvantage when the user is not satisfied with the fragrance of the laundry detergent contained therein. The user either must discard the unwanted laundry detergent or continue using such laundry detergent all the while being dissatisfied.

Additionally, when there are multiple members of a household, each member of the household may enjoy different fragrances for their laundry. It is, however, impractical for a family to purchase multiple containers of different fragranced laundry detergent causing some members of the family to be dissatisfied.

Therefore, there is a need for a fragrance delivery product that can infuse a fragrance into laundry to the specifications of one or more users taking into account the disadvantages of the prior art as set out herein.

### SUMMARY OF THE INVENTION

The inventive fragrance tablet and related method described herein satisfies the needs set out above.

In one aspect of the present invention, a fragrance tablet is disclosed for use during the washing of laundry. The fragrance tablet is dissolvable in wash water and when combined with non-fragranced laundry detergent, whether such detergent is powder, liquid or contained in a dissolvable gel pack, provides a desired scented laundry to the satisfaction of the user.

The term “fragranced” can mean any type of fragrance including synthetic fragrances, natural fragrances and plant oils; also known as essential oils. The use of fragrance is, therefore, not meant to be limiting, and those skilled in the art would recognize that such use is merely illustrative.

The term “fragranced tablet” can mean any type of fragrance delivery device suitable for incorporation with laundry detergent. The devices can include effervescent tablets, dissolvable gel packs, paste, powder, cream, liquid, slurry, etc. The use of fragranced tablets is, therefore, not meant to be limiting, and those skilled in the art would recognize that such use is merely illustrative.

The fragrance tablets can include various scents made from synthetic fragrances, natural fragrances and essential oils individually or in combination what are suitable for the needs and personal preferences of multiple users. Further, the fragrance tablet can include other components such as cleaning boosters such as enzymes, laundry boosters and fabric softener.

Upon reading the included description, various alternative embodiments will become obvious to those skilled in the art. These embodiments are to be considered within the scope and spirit of the subject invention, which is only limited by the claims which follow and their equivalents.

### BRIEF DESCRIPTION OF THE DRAWINGS

FIG. 1 is a schematic of the process to create an embodiment of the present invention.

### DESCRIPTION OF THE INVENTION

The present invention is generally directed toward fragrance tablet and more specifically toward fragrance table that combines with laundry detergent to create a desirable scent and related method.

An embodiment of the present invention includes a fragrance delivery device suitable for incorporation with laundry detergent for use during the washing of laundry. The tablet includes a scent made from natural fragrances and essential oils. The fragrance tablet is dissolvable in wash water and when combined with non-fragranced laundry detergent provides the desired scented laundry to the satisfaction of the user.

Variations of the fragrance delivery device includes effervescent tablets, dissolvable gel packs, paste, powder, cream, liquid, slurry, etc. Further, the fragrance can also be made from synthetic fragrances. An additional variation of this embodiment include the addition of other components such as cleaning boosters such as enzymes, laundry boosters and fabric softener.

An embodiment of the method of the present invention includes the combination of unscented laundry detergent and a fragrance tablet to create scented laundry to the satisfaction of the user. The fragrance tablet is capable of rapidly dissolving in water. In this embodiment, the fragrance tablet is an effervescent tablet.

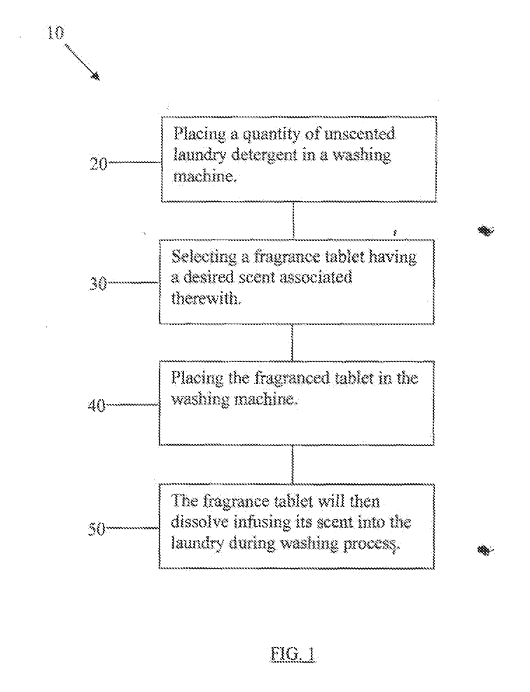

As illustrated in FIG. 1, an embodiment 10 of the method of the present invention includes placing a quantity of unscented laundry detergent in a washing machine 20, selecting a fragrance tablet having a desired scent associated therewith 30, and placing that fragranced tablet in the washing machine 40. The fragrance tablet will then dissolve infusing its scent into the laundry during washing process 50. A variation on this method is to first place the fragrance tablet into washing machine than then add the non-fragranced laundry detergent. Both of these methods can occur either before or after the wash water has started to fill the washing machine.

Another embodiment of the present invention is to combine two or more tablets having different scents to create a new scent.

### EXAMPLES

A first example illustrates a user having the ability to create different scents for her laundry using the same container of non-fragranced laundry detergent. In this example, the user is wanting to have different scents associated with the linens and the work clothes. She places the linens, a quantity of unscented laundry detergent in a washing machine along with a fragrance tablet having a first scent. The fragrance tablet quickly dissolves during the washing cycle impregnating the linens with its scent.

The user then places the work cloths, a second quantity of unscented laundry detergent in the washing machine and selects a fragranced tablet having a second scent. The work clothes will become impregnated with the second scent.

A second example illustrates creating different scents for specific family members using the same container of unscented laundry detergent. In this example, the mother can select a first fragrance tablet having a first scent for washing with a baby's clothes. The fragrance tablet for this load of laundry includes a natural fragrance that will not adversely affect the baby's skin. The remainder of the family's clothes can be washed using a second fragrance tablet having a second scent. The same unscented laundry detergent can be used for each of the different loads of laundry creating fragranced laundry detergent to the satisfaction of each member of the family.

While embodiments of the present invention have been illustrated and described, such disclosures should not be regarded as any limitation of the scope of our invention. The true scope of our invention is defined in the appended claims. Therefore, it is intended that the appended claims shall be construed to include both the preferred embodiment and all such variations and modifications as fall within the spirit and scope of the invention.

We claim:

1. A fragrance tablet comprising:
a fragrance selected from the group consisting of synthetic fragrances, natural fragrances and essential oils; and
a fragrance tablet delivery device, wherein said fragrance tablet delivery device is water soluble.

2. The fragrance tablet of claim 1 wherein said fragrance tablet delivery device is an effervescent tablet.

3. The fragrance tablet of claim 1 wherein said fragrance tablet delivery device is a gel pack.

4. The fragrance tablet of claim 1 further comprising a cleaning booster.

5. The fragrance tablet of claim 4 wherein said cleaning booster is an enzyme.

6. The fragrance tablet of claim 1 further comprising a laundry booster.

7. The fragrance tablet of claim 1 further comprising a fabric softener.

8. A method of creating a laundry detergent having a custom scent, said method comprising the steps of:
placing unscented laundry detergent into a clothes washer;
selecting a fragrance tablet having a desired scent; and
placing said selected fragrance tablet into said clothes washer.

9. The method of creating a laundry detergent of claim 8 wherein said step of placing unscented laundry detergent into a clothes washer occurs after said step of placing said selected fragrance tablet into said clothes washer.

10. The method of creating a laundry detergent of claim 8 wherein said step of selecting a fragrance tablet is further defined as selecting two or more fragrance tablets having a two or more desired scents.

11. A laundry detergent having a custom fragrance comprising:
unscented laundry detergent; and
a fragrance tablet having a scent associated with said fragrance tablet that is desirable to a a user, wherein said fragrance tablet infusing said fragrance into said unscented laundry detergent.

12. The laundry detergent of claim 11 wherein said fragrance tablet is dissolvable in said unscented laundry detergent.
